(19) United States
(12) Patent Application Publication
(10) Pub. No.: US 2015/0274644 A1
(43) Pub. Date: Oct. 1, 2015

(54) 14-HYDROXYEICOSANOIC ACID-BASED FATTY ACID AMIDE, AS AN ORGANOGELLING AGENT

(71) Applicant: ARKEMA FRANCE, Colombes (FR)

(72) Inventors: Michael Y. Bernard, Enghien Les Bains (FR); Christophe Duquenne, Paris (FR); Jean-Luc Dubois, Millery (FR)

(21) Appl. No.: 14/432,233

(22) PCT Filed: Oct. 2, 2013
(86) PCT No.: PCT/FR2013/052346

§ 371 Date: Mar. 30, 2015

(30) Foreign Application Priority Data
Oct. 5, 2012 (FR) 1259491

(51) Int. Cl. C07C 235/10; C08K 5/20; C07C 235/06

(52) U.S. Cl. CPC C07C 235/10; C07C 235/06; C08K 5/20

(57) ABSTRACT

The invention is directed to a 14-hydroxyeicosanoic acid-based fatty acid amide which may be a monoamide or a diamide, and to the use thereof as an organogelling agent or agent, also referred to as a rheology additive, in particular in coating, molding, mastic or sealing compositions or cosmetic compositions.

The invention relates to a 14-hydroxyeicosanoic acid-based fatty acid amide which may be a monoamide or a diamide, and to the use thereof as an organogelling agent or agent, also referred to as a rheology additive, in particular in coating, molding, mastic or sealing compositions or cosmetic compositions.

Fatty acid amides, and in particular diamides based on 12-hydroxystearic acid, are already known as organogelling agents, i.e. small organic molecules that are capable of gelling all kinds of organic solvents even at relatively low mass concentrations (less than 1% by mass) or as rheology additives, i.e. for modifying the rheology of an application formulation. They make it possible, for example, to obtain a thixotropic or pseudoplastic effect.

US 2011/251294 describes C₁₅ monoamide, monoamine or monoacid derivatives, which are in particular hydroxylated in position 12, as low molecular weight gelling agents for various applications in various solvents. Said document does not describe or suggest a 14-hydroxyeicosanoic acid amide, also referred to hereinbelow as 14-HEA or 14 HEA, or any performance associated with such a hydroxy acid.

EP 2 098 502 describes mono-, di- or tetraamides based on C₃ to C₂₀₀ monoamines or monoacids and more particularly diamides based on cyclic 1,2-cyclohexane diamine or diacid and, as function, on C₃ to C₂₀₀ monoacid or monoamine, which are suitable as vehicles for solid hot-melt inks, which undergo a phase change during printing at a temperature above the melting point of the solid ink. The role of these amides as vehicles for solid inks is to reduce the number of components and thus the cost of the ink and to make the solid ink composition uniform. No performance as an organogelling agent in solvent medium is described or suggested for these amides, nor is an amide based on 14-HEA described or suggested as such.

WO 2010/100939 describes a mixture of diamides of cycloaliphatic structure based on 1,2-cyclohexane diamine or diacid and on C₁₅ to C₂₁ monoamines or monoacids, as oil-gelling agents. Said document does not describe or suggest an amide based on 14-HEA as an organogelling agent.

U.S. Pat. No. 4,128,436 describes a diamide of oligomeric structure (oligoamide) based on hydroxystearic acid, a saturated aliphatic primary diamine and a C₂-C₁₀ diacid or a hydrogenated fatty acid dimer diacid, as a rheology control agent. No 14-HEA-based amide is described or suggested in particular as organogelling agent,

US 2012/129735 describes diamides based on a primary diamine and on a C₁₂-C₂₂ carboxylic monoacid, as a drilling fluid additive for oil wells, for controlling rheology, in particular for reducing the increase in viscosity at the head of drilling wells with a colder zone during the pumping of the extracted petroleum. No mention or suggestion of an organogelling agent based on an amide derived from 14-HEA is made.

The article entitled “Thermal Behavior of Prospective Hydroxy Acid Grease Thickeners” in JAOCS, 68, 3, 139-143 (1991) describes metal salts, in particular lithium salts, of fatty hydroxy acids and their thermal behavior and suggests, but without illustration and experimental proof of feasibility, their use as potential gelling agents for greases with a lubricating function. In point of fact, this suggestion is limited to only the metal salts examined regarding their thermal behavior, which differ from the behavior of the corresponding non-salified acids. No particular mention or suggestion of an amide based on 14-HEA as an organogelling agent is made.

12-Hydroxystearic acid, also referred to hereinbelow as 12HSA or 12-HSA, is already commonly used as a starting material for the preparation of fatty amides, However, this hydroxycarboxylic acid is derived from a channel whose sole source is castor oil. On account of the rapid development of certain applications widely using castor oil directly or in the form of its derivatives, its consumption has considerably increased, generating problems of availability and tension regarding the prices of these starting materials derived from the castor oil channel, such as 12-HSA. There is thus an increasing need to find an alternative solution to 12-HSA searching for new starting materials derived from a channel independent from castor oil, which channel is both abundant and also of renewable (or biosourced) origin, which can partially or totally, preferably totally, replace 12-HSA with satisfactory organogelling or rheological performance qualities. An organogelling agent is an additive, which, by definition, makes it possible to obtain a reversible gel in organic solvent medium. This is made possible by the specific molecular organization of the system resulting in a fine network structure, by specific interactions between the molecules of the organogelling agent, on the one hand, and by the interactions of said solvent with said molecule of the organogelling agent, on the other hand. The reversibility is obtained by modifying the shear rate and the temperature, such that increasing the shear rate and/or the temperature allows reversible destruction (by inverse variation) of this fine network structure.

The present invention is directed toward novel fatty amides that may be monoamides and/or diamides, preferably diamides, with a significantly reduced and preferably zero content of 12-hydroxystearic acid (12-HSA). This is performed by replacing 12-HSA partially and preferably totally with a specific saturated fatty hydroxycarboxylic acid: 14-hydroxyeicosanoic acid (also referred to hereinbelow as 14-HEA or 14HEA), derived from lesquerolic oil produced by extraction from Lesquerella seeds and thus from the cultivation of Lesquerella. This is achieved while maintaining entirely satisfactory performance qualities of the organogelling agent, in particular in terms of thixotropic power, and which may, in certain cases, be just as good as those of the organogelling agents of the prior art, for instance diamides exclusively based on 12-HSA and even better than certain organogelling agents derived from hydroxy acid isomers of 12-HSA. 14-Hydroxyeicosanoic acid is derived from lesquerolic oil extracted from Lesquerella seeds. It may be prepared by transesterification (with methanol) of said oil followed by hydrogenation of the transesterification product and finally hydrolysis of said hydrogenated ester to obtain 14-hydroxyeicosanoic acid. The methyl ester content of 14-HEA is enriched by selective liquid-liquid extraction of the mixture of methyl esters. Lesquerolic acid, present in ester form before hydrogenation, is 14-hydroxyeicosen-11-oic acid. A process for obtaining 14-hydroxy-eicosen-11-oic acid whose hydrogenation leads to 14-HEA is described in U.S. Pat. No. 3,057,893 and in particular in example 1. The partial or total replacement must therefore not affect the rheological performance qualities of the amide products obtained, while at the same time respecting a durable environment with starting materials of renewable origin. 14-Hydroxyeicosanoic acid prepared as cited above may contain among its impurities 12-hydroxystearic acid derived from the presence of ricinoleic acid in the Lesquerella oil used at the start for its preparation. No account is taken here of this very limited presence, generally less than 3% of 12-HSA associated with 14-HEA (on production of the 14-HEA), when the presence or absence of 12-HSA is considered, this presence or absence meaning the presence or absence of 12-HSA added in addition to that which is potentially residual and associated with the 14-HEA.

With this aim, the first subject of the present invention is a fatty acid amide, in particular a mono- and/or diamide, preferably a diamide, in which the acid 12-HSA is replaced, partially and preferably totally, i.e. to 100%, with 14-hydroxyeicosanoic acid (14-HEA). The term “total replacement” with 14-HEA means here that there is no addition of 12-HSA not associated with 14-HEA, since, as explained above, 14-HEA (as used) may contain as potential residual impurity a limited amount of 12-HSA. The amount of 12-HSA thus corresponds to the added amount not associated with 14-HEA. A particular option of this subject is a mixture of monoamide and of diamide, in particular with a major proportion of diamide.

The invention also relates to an organogelling agent and more particularly a rheology agent comprising said amide and to the use of said amide per se.

Finally, the invention also covers an organic binder composition comprising as rheology agent at least one amide according to the present invention.

Thus, the first subject of the invention is a fatty acid amide which comprises at least one product of reaction of a reaction mixture comprising:
a) at least one amine selected from:
   a linear aliphatic amine which may in particular be a C₂ to C₁₂, preferably C₂ to C₈ and more preferentially C₂ to C₆ monoamine and/or diamine, and/or
   a C₆ to C₁₈ and preferably C₆ to C₁₂ cycloaliphatic amine, and/or
   an aromatic amine that is preferably C₆ to C₁₂,
   said diamine a) preferably being a C₂ to C₁₂, in particular C₂ to C₈ and more preferentially C₂ to C₆ linear aliphatic amine,
b) 14-hydroxyeicosanoic acid in the absence or in the presence of 12-hydroxystearic acid,
c) optionally, at least one monoacid selected from C₆ to C₁₈, preferably C₆ to C₁₅ and more preferentially C₆ to C₁₂ non-hydroxylated saturated linear carboxylic acids,
d) optionally, at least a second amine different from a), which may in particular be a diamine and/or a monoamine, selected from C₂ to C₁₂, preferably C₂ to C₈ and more preferentially C₂ to C₆ linear aliphatic amines.

According to a first preferred possibility, said amide is a diamide and, in this case, said amines a) and d) are diamines.

However, the present invention also covers the case where said amide is a monoamide and, in this case, this monoamide may result from the reaction of a diamine and/or a monoamine according to a) with a monoacid b) and optionally a monoacid such as c) with, in the case of the reaction of a diamine, a total diamine/monoacid mole ratio of 1/1. More particularly, in the latter case (diamine), said monoamide is in reality a monoamide-amine. In this case, the reaction is performed by gradual addition of the monoacid b) (optionally with c)) to said diamine.

More preferentially, the monoamide according to the invention is derived from a monoamine according to a) and from 14-HEA according to b).

According to another option, said amide according to the invention may be a diamide and a monoamide, which means a mixture of diamide with a monoamide, preferably with said diamide being in major molar amount. Such a mixture of diamide and of monoamide may be obtained by using as amine a) and optionally as amine d) a mixture of a diamine with a monoamine.

According to a particular case, said 12-hydroxystearic acid may be present in said amide according to the invention, whether it is monoamide or diamide, and, in this case, the molar content of 14-hydroxyeicosanoic acid may range from 10% to 99%, preferably from 20% to 99% and more preferentially from 30% to 99% relative to the component b), i.e. all of the hydroxyl acids b).

More particularly, in said amide of the invention, monoamide and/or diamide and in particular diamide, said component b) is a mixture of 14-hydroxyeicosanoic acid and of 12-hydroxystearic acid (12HSA or 12-HSA), preferably with a mole ratio of 14-hydroxyeicosanoic acid (14-HEA) to 12-HSA ranging from 20/80 to 99/1 and more preferentially from 30/70 to 99/1.

According to another option, which is the most preferred, in said amide which may be monoamide or diamide, in particular diamide, 12-hydroxystearic acid is absent from said component b) and is totally replaced, i.e. to 100%, with 14-hydroxyeicosanoic acid.

According to a particular and preferred case, said amide is a diamide and said amines a) and optionally d), as defined above, are corresponding diamines. Said amide may in particular be a mixture of amides as products of reaction of the components as defined. It may correspond, for example, to a mixture of amines a) and d) and in particular to a mixture of diamine and of monoamine in a) and optionally in d). Thus, there may be, in the case of diamines a) and optionally of the diamines d) a mixture of diamides. In the case of monoamines a) and optionally d), there will be a mixture of monoamines and thus, consequently, of corresponding monoamides relative to the hydroxyl acid b) and optionally monoacid c). In the case of a mixture of diamines and of monoamines, there will be a mixture comprising several monoamides and several diamides corresponding to said amines a) and d).

According to the more particular option of said diamide, it bears two amide functions based on the same hydroxyl acid b) when 12-HSA is absent or based on two different hydroxy acids b) which are 14-HEA and 12-HSA, when 12-hydroxystearic acid is present in said diamide. The mixture of 14-hydroxyeicosanoic acid and of 12-hydroxystearic acid is not excluded from the scope of the invention in the case of a monoamide, in which case this is only possible in the form of a mixture of two monoamides that correspond to each hydroxy acid b), as cited, in addition to the possibility of mixture by the nature of the amine a) and optionally of the nature of the amine d).

According to another preferred option of the invention, said monoacid c) is present in a content such that the mole ratio of said hydroxy acid b) to said monoacid c) is from 1/2 to 4/1. Even more particularly, in the case where said amide of the invention is a diamide, it may bear an amide function based on a hydroxy acid b) and another function based on said monoacid c), which means that the mole ratio b/c is 1/1.

In general, the amine/acid (carboxy) mole ratio may range from 0.9 to 1.1 and preferably corresponds to the stoichiometric ratio of 1/1.

According to another possibility, said amide of the invention comprises at least two and preferably at least three different reaction products as derived from the reaction in said reaction mixture of an amine a) and possibly of an amine d) with a monoacid b) and possibly with a monoacid c). Thus, said amide, in the case where it is a diamide according to the invention, may be a mixture of two or three different reaction products in said mixture, of a diamine a) and possibly of a diamine d) with said monoacids b) and c) as described above. According to a more particular case of this possibility, said amide of the invention is a diamide and it may comprise a mixture of products having the following formulae:

b1-a1-c1

b1-a1-b1

and preferably:

b1-a1-c1

b1-a1-b1

b1-a2-b1

with a1: diamine residue according to a) bearing two amide groups —NHC(═O)—, a2: diamine residue according to d) bearing two amide groups —NHC(═O)—, b1: 14-hydroxyeicosanoic acid (14-HEA) residue without carboxyl group and c1: non-hydroxylated monoacid residue according to c) without carboxyl group.

According to another option, said amide according to the invention is a mixture of one or more diamides with one or more monoamides, said diamides in particular being in major molar amount with said amine a) and optionally said diamine d) being a mixture of a diamine with a monoamine. The term “a mixture of a diamine with a monoamine” means here and includes a mixture of one or more diamines with one or more monoamines.

As examples of linear aliphatic amines that are suitable and preferred for the amine component a) which is a diamine when said amide is a diamide, mention may be made of: ethylenediamine, propylenediamine, butylene (or tetramethylene)diamine, pentamethylenediamine, hexamethylenediamine, preferably ethylenediamine and hexamethylenediamine.

As monoamine amine components a), mention may be made of: ethylamine, propylamine, butylamine, pentylamine, hexylamine, ethanolamine, and preferably ethylamine, propylamine or hexylamine and ethanolamine.

As examples of cycloaliphatic diamines that are still suitable according to the amine component a) which is a diamine when said amide is a diamide, mention may be made of: cyclohexane -1,3-, -1,4- and -1,2- and in particular -1,3- or -1,4-diamine, isophoronediamine, bis(aminomethyl)-1,3-, -1,4- or -1,2-cyclohexane (derived from the hydrogenation, respectively, of m-, p- or o-xylylenediamine), preferably bis(aminomethyl)-1,3- or -1,4-cyclohexane, decahydronaphthalenediamine, bis(3-methyl, bis(4-aminocyclohexyl)methane (BMACM) or bis(4-aminocyclohexyl)methane (BACM), 1-{[4-(aminomethyl)cyclohexyl]oxy}propan-2-amine. The preferred cycloaliphatic diamines are chosen from: cyclohexane-1,3-, -1,4-diamine, bis(aminomethyl)-1,3-, -1,4- or -1,2-cyclohexane, isophoronediamine, bis(4-aminocyclohexyl)methane.

As examples of cycloaliphatic monoamine a) in the case of a monoamide amide, mention may be made of cyclohexylamine and isophorylamine.

As suitable and preferred examples of aromatic diamines as diamine amine component a) when said amide is a diamide, mention may be made of: m-, p-xylylenediamine, m-, p-phenylenediamine and m-, p-tolylenediamine.

As aromatic monoamine component a), mention may be made of: benzylamine, xylylamine and tolylamine. As examples of monoacids c), mention may be made of: hexanoic, heptanoic, octanoic, nonanoic, decanoic, undecanoic, dodecanoic, also known as lauric, or stearic acid. The following are preferred: hexanoic, octanoic, nonanoic and decanoic.

The second amine d), which is optional, is as defined for the linear aliphatic amines a) above, except that in this choice the amine d) is different from the amine a).

As regards component b), it is based on the specific hydroxy acid which is 14-hydroxyeicosanoic acid and may comprise as a mixture 12-hydroxystearic acid and preferably b) does not comprise any 12-hydroxystearic acid.

14-Hydroxyeicosanoic acid may be obtained from the seeds of oil-yielding plants of the Lesquerella genus.

The oil obtained predominantly contains triglycerides of lesquerolic acid, which is 14-hydroxy-11-eicosenoic acid. It also contains, but in minor amount, auricolic acid oil, which is 14-hydroxy-11,17-eicosadienoic acid. These two acids in oil form, i.e. in the form of triglycerides, after specific treatment and after hydrogenation, result in the same hydroxylated saturated fatty acid: 14-HEA. Depending on the species of the Lesquerella genus used as source of supply, the proportions of the fatty acids are variable and the oil may especially contain a residual portion of ricinoleic acid oil, which is 12-hydroxy-9-octadecenoic acid and of densipolic acid, which is 12-hydroxy-9,15-octadecadienoic acid. These last two hydroxylated fatty acid oils, at the end of the treatment and hydrogenation process, will potentially form a residual portion of 12-HSA that does not exceed 3% by weight of the final product recovered as the acid 14-HEA. It should be noted that this small percentage of residual 12-HSA associated with the 14-HEA is not considered in the acid defined according to b) when reference is made to the presence or absence of 12-HSA in said component b). This means, according to the invention, that the presence or absence of 12-HSA in b) is associated solely with the addition of 12-HSA and independently of the 14-HEA, i.e. independently of the residual content of 12-HSA in the 14-HEA used.

The conversion of lesquerolic oil into fatty acid fractions may be performed via a succession of steps whose number and sequence may vary.

The oil may thus undergo a first treatment of methanolysis, i.e. a transesterification in the presence of methanol. The methyl esters obtained are separated from the glycerol and subjected to a liquid-liquid extraction which makes it possible to enrich one of the phases with hydroxylated fatty acid methyl esters. This phase undergoes a hydrogenation to result in hydroxylated and saturated fatty methyl esters. During a final step, the esters are hydrolyzed to release the fatty chains in the form of free carboxylic fatty acids.

The methanolysis, hydrogenation and hydroxylated-fraction enrichment steps may be independent and may be inverted. Thus, the hydrogenation step may be performed directly on the oil, on the crude methyl esters, the enriched methyl esters, and also on the free fatty acids.

Moreover, in these processes, the enrichment step is optional depending on the degree of purity of the 14-HEA that it is desired to obtain.

Another subject of the invention relates to the use of the amides of the invention, in particular of the diamide, as organogelling agent, preferably as rheology agent or additive and more particularly in a preconcentrated composition in a plasticizer or in an organic solvent in preactivated paste form. In particular, this use relates to coating, bonding or adhesive, molding, mastic or sealing compositions or cosmetic compositions.

Another subject relates to an organogelling agent, preferably a rheology additive, which comprises at least one diamide as defined above according to the invention. More particularly, said organogelling agent is a rheology agent or additive, in particular a thixotropic agent or additive in a preconcentrated composition in an organic plasticizer or in an organic solvent in preactivated paste form.

The invention also covers an organogelling agent, preferably a rheology agent or additive, which comprises at least one diamide as defined above according to the invention, more particularly in a preconcentrated composition in an organic plasticizer or in an organic solvent in preactivated paste form. The preparation of such preconcentrated preactivated pastes may be performed according to the description of WO 1008/0 153 924.

Finally, the invention covers an organic binder composition, which comprises as rheology agent at least one amide as defined according to the present invention. More particularly, said organic binder is a binder for coating compositions selected from paints, varnishes, inks or gelled renderings or a binder for bonding or adhesive compositions or a binder for mastic or sealing agent or stripping agent compositions or for molding compositions or, finally, a cosmetic composition. Preferably, said binder is selected from epoxy resins, unsaturated and saturated polyesters, vinyl esters, alkyds, silane resins, polyurethanes, polyesteramides, solvent-based acrylic resins, i.e. resins in a non-reactive organic solvent medium, multi-functional acrylic monomers and/or oligomers or acryl acrylic resins with reactive diluents or inert resins diluted in a reactive or non-reactive solvent. The inert resins according to the invention are chlorinated or non-chlorinated elastomers or other chlorinated non-elastomeric polymers, for example based on vinyl chloride. The reactive solvent may be a monomer in which said resins are soluble and the non-reactive solvent may be an organic solvent for said resin which is chemically inert.

The molding compositions are in particular molding compositions for composites, including fiber-reinforced composites or compositions for molded parts, for example of SMC or BMC or laminated type, such as boat hulls or composite panels or for parts molded by casting or for molded parts with application of the composition by spraying with a gun or by brush or by roller.

These specific additives make it possible to modify the viscosity of the mastic, bonding, adhesive or coating compositions such as paints, varnishes, gelled renderings or inks or molding or sealing agent compositions or cosmetic compositions.

The amides according to the invention are in the form of micronized powder with a mean particle size ranging from 5 to 15 μm.

In order to be used as an organogelling additive and more particularly a thixotropic additive in an application composition such as a coating composition, for instance a paint, varnish, gelled rendering or ink or a bonding or adhesive composition or in a sealing agent or mastic composition or a molding composition or cosmetic composition, said amide needs to be activated in order to have its thixotropic nature. According to a first preferred option, this may be done independently of the final application composition in a preconcentrated composition of said amide in an organic plasticizer or in an organic solvent which is liquid at room temperature and suited to the amide and to the final application and preactivated in preactivated paste form, as described in WO 2008/0 153 924. In this case, this preactivated amide composition is added to the final application composition without the need for activation, insofar as said amide is added already preactivated with its “preactivated”, “preconcentrated” paste composition and suited to the final application. In this case, the final user, who is the formulator, will have no need to activate his formulation insofar as said preactivated amide thus supplemented gives this nature as soon as it is mixed into said final application composition.

Failing preactivation in preconcentrated preactivated paste form in a medium that is suited to (compatible with) the final application, the activation of said amide may be performed, according to a second option, in situ in the final application composition, but by the final user. The amide of the invention may thus be preactivated in preactivated and preconcentrated paste form.

This activation requires high-speed shear and corresponding heating with temperature rises that may range close to 120° C. depending on the products, and also a minimum necessary time, dependent on the temperature conditions and on the system, for developing final optimum rheological properties. These additives give the composition into which they are incorporated thixotropic behavior characterized by pronounced shear-thinning, i.e. a reduction in the viscosity when the shear increases, followed by a regain of viscosity that is dependent on the time (equivalent to a hysteresis effect). Thus, this type of additive gives the final composition excellent application properties that are characterized by high viscosity at rest, good stability of this viscosity on storage, good antisedimentation, ease of application and of extrusion following application and good sagging resistance once applied.

The fatty acid amide of the invention may be obtained by condensation (condensation reaction) between at least one primary amine according to a), the saturated fatty hydroxycarboxylic acid according to b) and optionally in the presence of a monocarboxylic acid according to c), optionally in the presence of a second primary diamine according to d), a), b), c) and d) being as defined above according to the invention. The reaction product may optionally be diluted in hydrogenated castor oil or (as a particular option) in hydrogenated lesquerolic oil, and, in this case (dilution in said oils), in a content that may range from 10% to 100% by weight relative to the total amide+hydrogenated castor oil or hydrogenated lesquerolic oil depending on the case, and preferably a content ranging from 20% to 100% by weight is used. The hydrogenated castor oil or the hydrogenated lesquerolic oil may be used to adapt the affinity of the final mixture (amide+hydrogenated castor oil or hydrogenated lesquerolic oil) relative to the composition of the final application formulation.

In the case of dilution in hydrogenated castor oil or hydrogenated lesquerolic oil, the addition takes place at a temperature of between 140 and 220° C. At the end of the addition, a solid mass is obtained, which is ground in powder form.

Said fatty acid amide may thus be used in powder or paste form preactivated as described above. The powder has a particle size of less than 100 μm and preferably less than 50 μm, and more preferentially at least 90% of said amide has a particle size of less than 20 μm and preferably less than 15 μm.

The examples described below in the experimental section are presented to illustrate the invention and its performance qualities and do not in any way limit the claimed scope.

### Experimental Section

### I—Starting Materials Used

TABLE 1
Starting materials used

| | Func- | Commercial | |
|---|---|---|---|
| Product | tion | reference | Supplier |
| 12-Hydroxystearic | Reagent | 12-HSA | Jayant Agro |
| acid | | | |
| Stearic acid | Reagent | 99% stearic acid | VWR |
| 9- and 10- | Reagent | See III-2 | |
| hydroxystearic acid | | | |
| 14-Hydroxy- | Reagent | See III-1 | |
| eicosanoic | | | |
| Hexamethylene- | Reagent | 98% hexamethylene- | Aldrich |
| diamine | | diamine | |
| Hexanoic acid | Reagent | 99% hexanoic acid | Aldrich |
| Ethylenediamine | Reagent | Ethylenediamine ≧ | Aldrich |
| | | 99.5% (GC) | |
| Epoxy resin | Binder | Araldite ® GZ | Huntsman |
| | | 7071X75 | |
| Epoxy resin | Binder | Araldite ® GY | Huntsman |
| | | 783 BD | |
| Degassing agent | De- | BYK ® A530 | Byk |
| | gassing | | |
| | agent | | |
| Dispersant | Disper- | Disperbyk ® 110 | Byk |
| | sant | | |
| Titanium dioxide | Pigment | Tiona ® 595 | Société des |
| | | | ocres de France |
| Iron oxide | Pigment | Bayferrox ® 915 | Lubrizol |
| Zinc phosphate | Pigment | ZP ® 10 | HEUCOPHOS |
| Talc | Additive | Finntalc ® MO5 | Mondo |
| | | | minerals |
| Silica | Filler | HPF6 | Sibelco |
| n-Butanol | Solvent | n-Butanol | Aldrich |
| Polyamide | Hardener | CRAYAMID ® 140 | Arkema |
| Xylene | Solvent | Xylene, reagent grade | Aldrich |

### II—Methods and Tests Used

The formulations were evaluated with two tests: the test of flow resistance (or sagging resistance) and an evaluation of the viscosity at various shear rates.

### Test of Flow Resistance

This is performed using a sagging controller (leveling/sagging tester from Sheen Instruments®) which makes it possible to establish the resistance of a coating to sagging due to gravity. This controller, made of stainless steel and equipped with a flat blade, comprises notches of increasing values.

The test consists in placing parallel strips of paint of different thicknesses onto a contrast strip by means of the sagging controller. The contrast card is immediately placed in a vertical position, the thinnest film at the top. The thickness at which the strips merge indicates the sagging tendency (resistance performance noted).

### Evaluation of the Viscosity

This is evaluated using a Brookfield® RV viscometer at 25° C. (spindle: S 4). The spindle speed is set at 50 rpm (revolutions per minute) and the viscosity of each paint is measured once this viscosity has stabilized. The operation is repeated for a speed of 20 rpm, 10 rpm, 5 rpm and 1 rpm.

### III—Preparation and Characterization of the Organogelling Agents and Rheology Additives

### III-1 Preparation of 14-HEA

A1) Preparation of the Hydrogenated Lesquerella Fatty Acids from Lesquerella Oil
Lesduerella Oil: Production Method from Lesquerella Seeds

The procedure on 25 kg of seeds (Techno)ogy Crops International) is as follows:
1. Flaking of the fresh Lesquerella seed on a flat-roll flattener.
2. The flakes are then dried for 16 hours at 100° C.
3. The flakes are placed in a percolation column.
4. A methanol/hexane mixture (50/50 by weight) is then circulated over the bed of flakes for 30 minutes at 40° C.
5. The mixture is then withdrawn and the bed of flakes is washed by 5 successive washes with the methanol/hexane mixture at 40° C. (5 minutes per wash).
6. The mixture is then evaporated under vacuum at 90° C. under 20 mbar for 5 minutes.
7. The oil and the gums are separated by centrifugation. The yield of oil is calculated on the basis of the mass of oil obtained relative to the expected theoretical weight of oil.
8. The oil is then washed until neutral by adding hot water and centrifugation, and is then dried under vacuum at 90° C. and 20 mbar for 5 minutes. The acid number and the composition of this oil are then measured.

TABLE 2
Analysis of the oil extracted with a methanol/hexane mixture

| Criteria | Method | |
|---|---|---|
| Acid number (mg KOH/g) | EN 14104 | 11.2 |
| Fatty acid profile | | |
| Palmitic (C16:0) | EN14105 | 1.6 |
| Palmitoleic (C16:1) | | 0.9 |
| Stearic (C18:0) | | 2.3 |
| Oleic (C18:1) | | 18.8 |
| Ricinoleic (C18:1-OH) | | 0.5 |
| Linoleic (C18:2) | | 10.1 |
| Densipoleic (18:2-OH) | | 0.2 |
| Linolenic and arachidic | | 11.6 (1) |
| Eicosenoic (C20:1) | | 0.9 |
| Lesquerolic (C20:1-OH) | | 52.1 |
| Auricolic (20:2-OH) | | 2.6 |
| Phospholipids (%) | Internal | 1.3 (2) |
| Corrected yield of oil (3), % | — | 106.5 |

(1) The two peaks are co-eluted. Linolenic acid is in major amount
(2) Calculated value; % phospholipids = % phosphorus × 26
(3) Corrected yield = yield of extraction oil − % phospholipids

The oil thus obtained is then refined by neutralization with sodium hydroxide and degummed with dilute phosphoric acid so as to remove the phospholipids. Finally, the oil is dried under vacuum. The oil obtained has the following characteristics:
Acid number: 0.5 mg KOH/g
Saponification number: 175 mg KOH/g
Hydroxyl number: 100 mg KOH/g
Iodine number: 95 g I₂/100 g
Lesquerolic acid content: 52%
Phosphorus content: 10 ppm
Content of water and volatiles: 0.1% by weight
Ash content: 0.1% by weight.
   Transesterification of the Oil with Methanol

In a first stage, a transesterification (with methanol) of the Lesquerella oil is performed, followed by hydrogenation and finally hydrolysis. An extraction step after the transesterification makes it possible to enrich the product in lesquerolic acid ester.

The methanolysis of the Lesquerella oil is performed with a methanol/oil mole ratio of 6 (i.e. twice the stoichiometric amount). The catalyst used is sodium methoxide in a content of 0.5% by weight and the reaction temperature is 60° C. The constituents are mixed together with vigorous stirring for 30 minutes. After methanolysis (transesterification) and removal of the glycerol by separation of the phases by settling, the esters are purified by washing with water and drying under vacuum. The specifications of the methyl esters are as follows:
Acid number: 0.5 mg KOH/g
Saponification number: 175 mg KOH/g
Iodine number: 95 g I₂/100 g
Content of residual glycerides (analysis by GC): 1.9% by weight
Content of lesquerolic acid (methyl ester): 52%
   A2) Preparation of a Mixture Enriched in 14-Hydroxyeicosanoic Acid from Methyl Esters as Described in the Preceding Paragraph A1

The mixture of esters derived from the transesterification step is subjected to a step of liquid-liquid extraction with a methanol/hexane mixture. In the practical implementation of the example, the methanol contains 5% by weight of water. The non-hydroxylated fatty acids are more compatible with the hexane phase, whereas the hydroxylated fatty acids such as lesquerolic acid are more compatible with the methanol phase. Hexane was used as apolar solvent and the polar solvent consisted of hydrated methanol. A sequence of depletion and enrichment steps is performed.
1. 5 g (methyl ester of Lesquerella oil)+30 ml of apolar solvent+15 ml of polar solvent are stirred for 5 minutes in a separating funnel and give a heavy phase PL1+light phase pI1.
2. The light phase pI1 is taken up in 15 ml of polar solvent and again gives a heavy phase PL2 and a light phase pI2.
3. The heavy phase PL1 and the heavy phase PL2 are taken up in 30 ml of apolar solvent and again give a heavy phase PL3 and a light phase pI3.
4. The heavy phase PL3 is taken up in 30 ml of apolar solvent to give a heavy phase PL4 and a light phase pI4.
   The recovered fractions are then concentrated, by evaporation of the solvents.
1. The heavy phase PL4 gives the polar fraction.
2. The light phases pI2+pI3+pI4 are combined to give the apolar fraction.

TABLE 3
Analytical balance of the esters derived from the extraction

| | Starting | | |
|---|---|---|---|
| | material | Heavy phase | Light phase |
| Polar solvent | | Methanol 95% | Methanol 95% |
| Apolar solvent | | Hexane | Hexane |
| Mass yld*, % | | 16.5 | 83.5 |
| Methyl lesquerolate extraction | | 25.4 | 74.8 |
| yld*, % | | | |
| Acid number | 0.72 | nd | 1.12 |
| Me C16:1 (%) | 0.5 | 0.1 | 0.6 |
| Me C16 (%) | 1.3 | 0.1 | 1.6 |
| Me C18:2 (%) | 9.1 | 0.6 | 11.7 |
| Me C18:1 (%) | 23.5 | 0.3 | 27.5 |
| Me C18:0 (%) | 1.8 | 0.1 | 2.2 |
| Me C20:0 (%) | 0.9 | 0.0 | 1.1 |
| Me C20:1 (%) | 1.0 | 0.0 | 0.7 |
| Me C18:1-OH (%) | 0.3 | 0.2 | 0.3 |
| Me C20:1-OH (%) | 60.3 | 92.9 | 54.0 |
| Monoglyceride (%) | 1.7 | 5.8 | 0.8 |
| Diglyceride (%) | 0.1 | 0.0 | 0.1 |
| Triglyceride (%) | 0.0 | 0.0 | 0.0 |

*Yld: yield

### Hydrogenation of the Enriched Methyl Ester

A hydrogenation and a hydrolysis are performed on the fraction enriched in lesquerolic acid (in Me ester form) to give a mixture rich in 14-hydroxyeicosanoic acid (hydrogenation and hydrolysis described below).

For the hydrogenation in an autoclave, a catalyst of Raney nickel type sold by Johnson Matthey is used, at a content of 0.5% by weight. The hydrogenation temperature is 150° C. at a hydrogen pressure of 8 bar. This step leads to a product with an iodine number of 3 g I2/100 g.

### Saponification of the Hydrogenated Methyl Ester

Finally, a saponification (hydrolysis) step is performed by adding sodium hydroxide, followed by an acidification step with sulfuric acid. The resulting mixture is washed with water, the phases are separated by settling and the product is dried under vacuum.

The characteristics of the mixture obtained are:
Acid number: 1 mg KOH/g
Hydroxyl number: 145 mg KOH/g
Iodine number: 3 g I₂/100 g
Content of 14-hydroxyeicosanoic acid: 89%.

### III-2 Preparation of a Mixture of 9-Hydroxy and 10-Hydroxystearic Acids (9-HSA and 10-HSA)

The preparation is based on the hydroxylation of the double bond of a fatty acid (transposed here to oleic acid) as described in “Addition of Formic acid to Oleifinic compounds” by H. B. Knights, R. E. Koos and Daniel Swern, May 2, 1953.

Other methods may be used for gaining access to the monohydroxylated fatty acids 9- and 10-HSA.

### Preparation Method Used

319.1 grams of oleic acid, 677.6 grams of formic acid and 3.3 grams of perchloric acid are placed, under a nitrogen atmosphere, in a 1 liter round-bottomed flask equipped with a thermometer, Dean-Stark apparatus, a condenser and a stirrer. After 30 minutes at reflux, the excess formic acid is evaporated off under vacuum at 75 mbar and 65° C.

The compound obtained (102 grams) is then hydrolyzed with a 6N sodium hydroxide solution (100 grams). Finally, the product is neutralized by slow addition of fuming hydrochloric acid (64 grams) in 66 grams of water.

The purification is performed by dissolving the reaction medium in toluene and by three successive washes with 11% NaCl solution. The toluene is then evaporated off and the product is recrystallized from hexane. 27 grams of a mixture of monohydroxylated fatty acids 9- and -10 HSA are thus obtained.

### III-3 Preparation of Amides According to the Invention and Comparative Amides

### EXAMPLE 1

### Comparative Diamide A (Based on Stearic Acid)

49.96 grams of hexamethylenediamine (i.e. 0.43 mol, 0.86 equivalent amine) and 244.65 grams (0.86 mol, 0.86 equivalent) of stearic acid are placed, under a nitrogen atmosphere, in a 1 liter. round-bottomed flask equipped with a thermometer, Dean-Stark apparatus, a condenser and a stirrer.

The mixture is heated to 200° C. still under a stream of nitrogen. The water removed begins to accumulate in the Dean-Stark apparatus from 150° C. The reaction is monitored by the acid number and the amine number. When the acid and amine values (numbers) are less than 10 mg KOH/g, the reaction mixture is cooled to 150° C. and then discharged into a silicone-treated mold. Once cooled to room temperature, the product is micronized mechanically by milling and screening to obtain a fine and controlled particle size with a mean size obtained of 7 μm.

### EXAMPLE 2

### Comparative Diamide B (Based on 9- and 10-Hydroxystearic Acids)

49.96 grams of hexamethylenediamine (i.e. 0.43 mol, 0.86 amine equivalent), 260.48 grams of a mixture of 9- and 10-hydroxystearic acids (i.e. 0.86 mol, 0.86 equivalent acid) as described in paragraph III-2 are placed, under a nitrogen atmosphere, in a 1 liter round-bottomed flask equipped with a thermometer, Dean-Stark apparatus, a condenser and a stirrer. The mixture is heated to 200° C. still under a stream of nitrogen. The water removed accumulates in the Dean-Stark apparatus from 150° C. The reaction is monitored via the acid number and the amine number. When the acid and amine values are less than 10 mg KOH/g, the reaction mixture is cooled to 150° C. and then discharged into a silicone-treated mold. Once cooled to room temperature, the product is micronized mechanically as in Example 1 with the same mean size.

### EXAMPLE 3

### Comparative Diamide C (Based on 12 HSA)

49.96 grams of hexamethylenediamine (i.e. 0.43 mol, 0.86 amine equivalent) and 271.04 grams of a mixture of 12-hydroxystearic acids (i.e. 0.86 mol, 0.86 acid equivalent) are placed, under a nitrogen atmosphere, in a 1 liter round-bottomed flask equipped with a thermometer, Dean-Stark apparatus, a condenser and a stirrer. The mixture is heated to 200° C. still under a stream of nitrogen. The water removed accumulates in the Dean-Stark apparatus from 150° C. The reaction is monitored via the acid number and the amine number. When the acid and amine values are less than 10 mg KOH/g, the reaction mixture is cooled to 150° C. and then discharged into a silicone-treated mold. Once cooled to room temperature, the product is micronized mechanically as in Example 1 with the same mean size.

### EXAMPLE 4

### Diamide D According to the Invention

49.96 grams of hexamethylenediamine (i.e. 0.43 mol, 0.86 amine equivalent) and 307.30 grams of a mixture enriched in 14-hydroxyeicosanoic acid (i.e. 0.86 mol, 0.86 acid equivalent) as described above (final product A2 containing 89% 14-HEA) are placed, under a nitrogen atmosphere, in a 1 liter round-bottomed flask equipped with a thermometer. Dean-Stark apparatus, a condenser and a stirrer. The mixture is heated to 200° C. still under a stream of nitrogen. The water removed accumulates in the Dean-Stark apparatus from 150° C. The reaction is monitored via the acid number and the amine number. When the acid and amine values are less than 10 mg KOH/g, the reaction mixture is cooled to 150° C. and then discharged into a silicone-treated mold. Once cooled to room temperature, the product is micronized mechanically as in Example 1 with the same mean size.

### EXAMPLES 5, 6, 7 and 8

### Comparative Diamide E (Ex. 5), Comparative Diamide F (Ex. 6), Comparative Diamide G (Ex. 7) and Diamide H According to the Invention (Ex. 8)

The same procedure was used as in Examples 1 to 4, but with the following reaction components presented in the table below:

TABLE 4
Composition of the diamides compared

| Example | Reagent | Moles | Equivalents |
|---|---|---|---|
| 1 | Hexamethylenediamine | 0.43 | 0.86 |
| Comparative | Stearic acid | 0.86 | 0.86 |
| diamide A | | | |
| 2 | Hexamethylenediamine | 0.43 | 0.86 |
| Comparative | 9- and 10-hydroxystearic | 0.86 | 0.86 |
| diamide B | acids | | |
| 3 | Hexamethylenediamine | 0.43 | 0.86 |
| Comparative | 12-hydroxystearic acid | 0.86 | 0.86 |
| diamide C | | | |
| 4 | Hexamethylenediamine | 0.43 | 0.86 |
| Diamide D | 14-hydroxyeicosanoic | 0.86 | 0.86 |
| according to the | acids | | |
| invention | | | |
| 5 | Ethylenediamine | 0.5 | 1 |
| Comparative | Hexanoic acid | 0.5 | 0.5 |
| diamide E | Stearic acid | 0.5 | 0.5 |
| 6 | Ethylenediamine | 0.5 | 1 |
| Comparative | Hexanoic acid | 0.5 | 0.5 |
| diamide F | 9- and 10-hydroxystearic | 0.5 | 0.5 |
| | acids | | |
| 7 | Ethylenediamine | 0.5 | 1 |
| Comparative | Hexanoic acid | 0.5 | 0.5 |
| diamide G | 12-hydroxystearic acid | 0.5 | 0.5 |
| 8 | Ethylenediamine | 0.5 | 1 |
| Diamide H | Hexanoic acid | 0.5 | 0.5 |
| according to the | 14-hydroxyeicosanoic | 0.5 | 0.5 |
| invention | acids | | |

### III-4 Organogelling Properties for Diamides A, B, C, D

20 grams of diamide A of Example 1 (comparative) ground beforehand (mean size, d50=10 μm) and 80 grams of xylene are placed in a metal dish at room temperature. Using a Dispermat® CV disperser equipped with a paddle 4 cm in diameter, the two products are mixed at a speed of 2000 revolutions/minute (or rpm) for 30 minutes at a temperature not exceeding 20° C., by regulating the temperature via circulation of cold water.

Activation: The dish is then carefully closed and placed in an oven preheated to 65° C. for 24 hours. Once cooled and after 4 hours at rest, the appearance of the mixture is observed. The same procedure is performed on the diamides of Examples 2 (B comparative), 3 (C comparative), 4 (D according to the invention). The results are presented in table 5.

TABLE 5
Appearance of the mixtures after activation

| Amide according | |
|---|---|
| to example | Appearance |
| 1 (A) | Liquid |
| 2 (B) | Soft paste but allows a wooden spatula to |
| | remain vertical |
| 3 (C) | Hard paste allowing a metal spatula to |
| | remain vertical |
| 4 (D) | Hard paste allowing a metal spatula to |
| | remain vertical. |
| | Thicker consistency than C of Example 3. |

The results show that the invention is, after the step of activation and cooling, in the form of a hard paste which allows a metal spatula to remain vertical in the paste. This result perfectly illustrates the capacity of the diamide D of Example 4 according to the invention to be an organogelling agent, in contrast with amide A of Example 1. Furthermore, the consistency of the gel obtained is much better with amide D of Example 4 based on 14-HEA than amide B of Example 2 based on 9- and 10-HSA and is at least just as good as amide C of Example 3 based on 12-HSA as targeted at the start.

### IV—Evaluation of the Rheological Performance Qualities in a Paint Formulation

### Paint Formulations Used for the Evaluation

### 1—Preparation

A “millbase” formulation is prepared with the proportions in table 3 in the following manner:

In a dispersing bowl (Dispermill 2075 yellow line, supplier: Erichsen®) heated via a jacket system:

1. Introduction of the epoxy binders and also the dispersant and the degassing agent. Homogenization takes place after 2 minutes at 800 revolutions/minute.
2. Introduction of the fillers and pigments, followed by milling at 3000 revolutions/minute for 30 minutes using a 7 cm paddle. By means of the jacketed bowl, this step is cooled with a bath of cold water (20° C.).
3. Introduction of the solvents.

### 2—Activation

24 hours after preparing the millbase, the formulation is again dispersed at 3000 revolutions/minute using a 4 cm paddle. The diamide to be tested is introduced into the millbase at a given activation temperature (ranging from 40° C. to 70° C.) for 20 minutes at 3000 revolutions/minute.

After adding the hardener diluted in the millbase, the paints are adjusted with a xylene/butanol mixture (1/1) to a working viscosity of 0.4 Pa·s, measured on cone 4 at 25° C. at 2500 s⁻¹ using a Brookfield® CAP 1000 viscometer. The proportions between the hardener and the solvent mixture are defined in table 6.

After the adjustment, the paint is mixed at 1500 revolutions/minute for 2 minutes, and then left to stand for 30 minutes.

TABLE 6
“Millbase” formulation

| Millbase composition | Function | mass % |
|---|---|---|
| Araldite ® GZ 7071X75 | Binder | 17.3 |
| Araldite ® GY 783 BD | Binder | 12.9 |
| BYK ® A530 | Degassing agent | 0.5 |
| Disperbyk ® 110 | Dispersant | 0.5 |
| Tiona 595 (Titanium dioxide) | Pigment | 1.9 |
| Bayferrox ® 915 595 | Pigment | 4.1 |
| (iron oxide) | | |
| ZP ® 10 (zinc phosphate) | Pigment | 7.5 |
| Finntalc ® MO5 | Filler | 9.4 |
| Silice HPF6 | Filler | 19.0 |
| n-Butanol | Solvent | 5.4 |
| Diamide | Rheology | 0.8 |
| | additive | |
| TOTAL | | 79.3 |

TABLE 7
Hardener

| Composition of the hardener | mass % |
|---|---|
| Crayamid ® 140 | 8.8 |
| Xylene | 11.9 |
| TOTAL | 20.7 |

### 3—Evaluation of the Rheology of the Formulations and Results (see Tables 8 and 9)

Various paint formulations were prepared according to the proportions in table 3 and 4 and with various activation temperatures ranging from 40 to 70° C. according to the protocol mentioned above.

The sagging resistance and rheology results show that diamide H of the invention based on 14-HEA has:
a better sagging resistance (table 8) than diamide F based on 9- and 10-HSA and just as efficient as the diamide based on 12-HSA at an activation temperature of 70° C. in this formulation. It thus has the characteristics necessary for a rheology additive;
a thixotropic effect on the formulation once it is activated above 40° C., unlike diamide E (based on stearic acid) which is inactive irrespective of the temperature (table 9) and diamide H according to the invention is better than diamide F based on 9- and 10-hydroxystearic acid.

TABLE 8
Sagging resistance results

| | | Sagging resistance |
|---|---|---|
| Test | Diamide | (μm) |
| Example 5, 40° C. | E | 225-250 |
| Example 5, 50° C. | E | 225-250 |
| Example 5, 60° C. | E | 225-250 |
| Example 5, 70° C. | E | 225-250 |
| Example 6, 40° C. | F | 225-250 |
| Example 6, 50° C. | F | 225-250 |
| Example 6, 60° C. | F | 375-400 |
| Example 6, 70° C. | F | 400-425 |
| Example 7, 40° C. | G | 550-575 |
| Example 7, 55° C. | G | 550-575 |
| Example 7, 70° C. | G | >750 |
| Example 7, 80° C. | G | >750 |
| Example 8, 40° C. | H | 400-425 |
| Example 8, 55° C. | H | 400-425 |
| Example 8, 70° C. | H | >750 |
| Example 8, 80° C. | H | >750 |

TABLE 9
Rheological results

| | | Brookfield viscosity at 25° C. (mPa · s) | | | | |
|---|---|---|---|---|---|---|
| | | 1 | 5 | 10 | 50 | 100 |
| Test conditions | Diamide | RPM | RPM | RPM | RPM | RPM |
| Example 4, 40° C. | E | 2000 | 1150 | 906 | 657 | 588 |
| Example 5, 50° C. | E | 1912 | 1002 | 858 | 642 | 580 |
| Example 5, 60° C. | E | 1970 | 1055 | 880 | 640 | 584 |
| Example 5, 70° C. | E | 1989 | 1107 | 900 | 654 | 588 |
| Example 6, 40° C. | F | 2000 | 1120 | 920 | 660 | 596 |
| Example 6, 50° C. | F | 2400 | 1280 | 1020 | 700 | 630 |
| Example 6, 60° C. | F | 9600 | 3400 | 2300 | 1092 | 862 |
| Example 6, 70° C. | F | 11800 | 4000 | 2640 | 1196 | 922 |
| Example 7, 40° C. | G | 17600 | 5360 | 3420 | 1456 | 1076 |
| Example 7, 55° C. | G | 18200 | 5600 | 3600 | 1528 | 1128 |
| Example 7, 70° C. | G | 30400 | 8680 | 5280 | 2040 | 1426 |
| Example 7, 80° C.. | G | 30800 | 8360 | 5360 | 2064 | 1456 |
| Example 8, 40° C. | H | 8600 | 2880 | 1960 | 972 | 772 |
| Example 8, 55° C. | H | 9800 | 3280 | 2240 | 1064 | 832 |
| Example 8, 70° C. | H | 22000 | 6480 | 4060 | 1644 | 1182 |
| Example 8, 80° C. | H | 23000 | 6480 | 4280 | 1776 | 1280 |

1. A fatty acid amide comprising at least one product of reaction of a reaction mixture comprising:
a) at least one amine selected from:
a linear aliphatic amine, and/or
a C₆ to C₁₈ cycloaliphatic amine, and/or
an aromatic amine
b) 14-hydroxyeicosanoic acid (14-HEA) in the absence or in the presence of 12-hydroxystearic acid (12-HSA),
c) optionally, at least one monoacid selected from C₆ to C₁₈, non-hydroxylated saturated linear carboxylic acids,
d) optionally, at least a second amine different from a), selected from C₂ to C₁₂ amines.

2. The amide as claimed in claim 1 wherein said 12-hydroxystearic acid is present and the molar content of 14-hydroxyeicosanoic acid is from 10% to 99% of said component b).

3. The amide as claimed in claim 1 wherein said component b) is a mixture of 14-hydroxyeicosanoic acid (14-HEA) and 12-hydroxystearic acid (12-HSA).

4. The amide as claimed in claim 1 wherein said 12-hydroxystearic acid is absent from said component b) and is replaced with 14-hydroxyeicosanoic acid.

5. The amide as claimed in claim 1 wherein said amide is a monoamide and said amines a) and d) are diamines and/or monoamines.

6. The amide as claimed in claim 1 wherein said amide is a diamide and said amines a) and d) are diamines.

7. The amide as claimed in claim 6, wherein said diamide bears two amide functions based on the same hydroxy acid b) when 12-HSA is absent or based on two different hydroxy acids b), 14-HEA and 12-HSA, when 12-HSA is present.

8. The amide as claimed in claim 6 wherein in said diamide, said monoacid c) is present such that the mole ratio of said hydroxy acid b) to said monoacid c) is from 1/2 to 4/1.

9. The amide as claimed in claim 6 wherein said diamide bears an amide function based on a hydroxy acid according to b) and another function based on said monoacid c) corresponding to a mole ratio b)/c) of 1/1.

10. The amide as claimed in claim 1 comprising at least two different reaction products, as derived from the reaction in said reaction mixture of an amine a) or d) with a monoacid b) or c).

11. The amide as claimed in claim 10, wherein it is a diamide comprising a mixture of products having the following formulae:
b1-a1-c1
b1-a1-b1
with a1: diamine residue according to a) bearing two amide groups —NHC(═O)—, a2: diamine residue according to d) bearing two groups —NHC(═O)—, b1: residue of the acid 14-HEA without a carboxyl group and c1: non-hydroxylated monoacid residue according to c) without a carboxyl group.

12. The amide as claimed in claim 1 wherein it is a mixture of one or more diamides with one or more monoamides, with said amine a) and optionally said diamine d) being a mixture of a diamine with a monoamine.

13. (canceled)

14. (canceled)

15. An organogelling agent comprising at least one diamide according to claim 6.

16. The organogelling agent as claimed in claim 15, wherein it is a rheology agent or additive in a composition preconcentrated in an organic plasticizer or in an organic solvent, in preactivated paste form.

17. An organic binder composition, comprising as rheology agent at least one amide according to claim 1.

18. The organic binder composition of claim 17, wherein said organic binder is a binder for coating compositions selected from paints, varnishes, inks and gelled renderings or a binder for bonding or adhesive compositions or a binder for mastic or sealing agent or stripping agent compositions or for molding compositions or cosmetic compositions.

19. The composition of claim 17 wherein said binder is selected from the group consisting of: epoxy resins, unsaturated and saturated polyesters, vinyl esters, alkyds, silane-based resins, polyurethanes, polyesteramides, solvent-based acrylic resins, multifunctional acrylic monomers and/or oligomers or acryl acrylic resins with reactive diluent or inert resins diluted in a reactive or non-reactive solvent.

20. An amide according to claim 1, wherein said amine a) is a linear aliphatic amine selected from diamine and/or monoamine in C₂ to C₁₂.

21. An amide according to claim 20, wherein said amine a) is in C₂ to C₆.

22. An amide according to claim 1, wherein said monoacid c) is in C₆ to C₁₅.

23. An amide according to claim 22, wherein said monoacid c) is in C₆ to C₁₂.

24. An amide according to claim 1, wherein said second amine d) is in C₂ to C₈.

25. An amide according to claim 1, wherein said second amine d) is in C₂ to C₆.
